(19) United States
(12) Patent Application Publication
(10) Pub. No.: US 2005/0273110 A1
(43) Pub. Date: Dec. 8, 2005

(54) Devices for performing fusion surgery using a split thickness technique to provide vascularized autograft

(76) Inventors: Frank H. Boehm JR., Utica, NY (US); Benedetta Delorenzo Melnick, Rome, NY (US)

Correspondence Address: DILWORTH & BARRESE, LLP, 333 EARLE OVINGTON BLVD., UNIONDALE, NY, 11553, US

(21) Appl. No.: 11/128,851

(22) Filed: May 12, 2005

Related U.S. Application Data
(60) Provisional application No. 60/570,431, filed on May 12, 2004.

(51) Int. Cl. A61B 17/32

(52) U.S. Cl. 606/84

(57) ABSTRACT

A technique for providing vascularized autograft to a fusion site in the posteriolateral aspect of a lumbar fusion mass by using a split-thickness laminoplasty technique, is disclosed in co-pending U.S. Provisional Application 60/379,371 filed on May 10, 2002 and U.S. patent application Ser. No. ______ filed concurrently with the present application. Devices used to accomplish osteotomies of the laminae, facet joints, and transverse processes are disclosed. The devices include a shaft that can guide the devices, a method of deploying a wire bone saw for dividing these structures through a coronal plane, leaving the periosteum, musculature, and hence blood supply intact. This novel and non-obvious technique will allow for the application of vascularized autograft to the fusion bed in posterior or posteriolateral fusion of the spine.

### CROSS REFERENCE TO RELATED APPLICATIONS

This application claims priority under 35 U.S.C. 119(e) to U.S. Provisional Application Ser. No. 60/379,371 filed on May 10, 2002, U.S. Provisional Application Ser. No. 60/470,167 filed on May 12, 2003, and U.S. Provisional Application Ser. No. 60/570,431 filed on May 12, 2004, the entire contents of each of which are incorporated herein by reference.

### BACKGROUND OF INVENTION

1. Field of Invention

The present invention relates to a set of devices utilized in conjunction with a surgical procedure and method to provide vascularized autograft to a fusion mass in a surgical fusion procedure of vertebra of the lumbar spine utilizing a posteriolateral approach.

2. Discussion of the Related Art

It has long been accepted that even in the best of hands, the process of fusing two or more of the lumbar vertebrae using a posteriolateral approach has an inherent rate of success on the order of 60-70%. Fusion is the joining together of adjacent vertebrae of the spine by establishing a milieu in which new bone growth ultimately occurs connecting the adjacent vertebrae. Techniques that have been proposed to accomplish this include establishing that milieu in the disc space (also known as interbody fusion) as well as establishing that milieu in the posteriolateral aspects of the spinal structures, including the laminae, the facet joints, and the transverse processes (also known as posterior/posteriolateral mass fusions). Measures, which are proven to increase this rate of success, include stabilization with spinal instrumentation, use of autograft as opposed to allograft, use of Bone Morphogenic Protein (BMP), and use of electronic bone growth stimulation devices. In each of these instances, different strategies are employed to attempt to promote healing and maturation of the fusion site.

The use of electronic stimulation devices is thought to assist in the process of promoting bony fusion by altering the local pH and acid-base balance to create a milieu in which Wolfe's Law would apply, thus promoting the migration of osteoblasts and the secretion of calcium and other substances favorable to bone formation. Both internally-implantable and externally applied systems have been shown to be successful in this setting.

The use of spinal instrumentation has been clearly shown to enhance the rate at which spinal fusions mature. Various types of spinal instrumentation systems include rods connected to the laminae by hooks, rods and/or plates, which are connected to the vertebrae by screws secured to the facets or pedicles, or fusion cages passed into the intervertebral spaces. The rationale that substantiates the use of spinal instrumentation suggests that the efficacy of these techniques rests in their ability to reduce/eliminate motion between the vertebrae at the proposed site of the fusion, as well as maintaining the osseous structures in close proximity.

The use of Bone Morphogenic Protein (BMP) to enhance fusion rates has held theoretical attraction for some time now. The role postulated for this osteogenic protein includes enhancement of the activity of osteoblasts and more rapid deposition of osteogenic substrates. Recently, the initial clinical use of BMP has confirmed the clinical utility thereof, but its final role in spinal fusion technology remains to be clarified further.

Many surgeons still believe that the use of autograft, defined as bone harvested from the patient in whom the fusion is being performed, provides superior results when compared to fusions utilizing allograft, defined as bone from other sources, generally cadaveric donors. However, this bone is generally harvested from a remote site, such as the hip, necessitating a separate surgical procedure, with its own attendant risks and morbidity. Outcomes, including prolonged pain at this surgical site, are well known. Although there are data to suggest that the fusion rates in cases utilizing autograft are higher, it has been demonstrated that this is sometimes offset when the morbidity at the donor sites is taken into consideration.

It has been shown that in bone fusion at other sites, the use of vascularized autograft can significantly increase the rate of fusion as well as reduce the length of time to maturation of fusion. There have been case reports discussing the utility of vascularized fibular struts in the cervical spine, but they have not been found to be pragmatic for general use, and its application to the lumbar spine has not been reported.

Therefore, a need exists for devices and tools that would facilitate a method of providing vascularized autograft to a fusion site during posteriolateral fusion of the spine. This would provide a greater opportunity for healing and maturation of the fusion, and would provide additional benefit to the patient insofar that a separate surgical site for harvesting allograft would not be necessary.

### SUMMARY OF THE INVENTION

It is, therefore, an object of the present invention to provide a set of tools and devices that would allow a surgical procedure to be performed in which vascularized autograft can be harvested from the spine using a minimally traumatic procedure.

It is a further object of the present invention to provide a set of tools and devices that would allow the vascularized autograft which has been harvested to be utilized in the fusion bed during a posterior or posteriolateral fusion of the spine.

It is a further object of the present invention to provide a set of tools and devices that would allow for the division of the lamina, facets and transverse processes in the coronal plane, thus preserving the muscle and periosteal attachments to these bony structures and creating the vascularized autograft needed to fulfill the objectives enumerated above.

It is yet another object of this invention to provide a set of tools and devices that would prevent and protect from injury other structures in and near the spine.

The above and other objects of this invention can be achieved by the provision of the inventive tools and devices that can be used in a fusion procedure utilizing a split-thickness laminoplasty, created by using the tools to divide the lamina of the spine in a coronal plane, allowing the posterior portion of the split-thickness laminoplasty to remain connected to the muscles, thus creating a vascularized autograft for fusion in posteriolateral fusion techniques of the spine.

In accordance with one aspect of the invention, using the inventive tools and devices a surgeon can perform an osteotomy of the facet joints of the spine in the coronal plane, to allow for the posterior aspect of the facet joints to remain attached to the muscle, thus creating a vascularized autograft for use in fusion of the spine using a posteriolateral technique.

In accordance with another aspect of the invention, using the inventive tools and devices a surgeon can perform an osteotomy of the transverse processes of the spine in the coronal plane, to allow both portions of the osteotomized transverse process to provide a vascularized autograft for use in fusion of the spine using a posteriolateral technique.

The inventive tools and devices comprise a shaft, which can be used by the surgeon to guide and direct the tool to the surgical site; a rod coaxially disposed within the shaft and traversing the length of the shaft, which is engaged at one end to a handle mechanism and is engaged at another end with a gear mechanism that translates actuation of the handle mechanism into actuation of a blade mechanism; and a cartridge which houses the blade mechanism.

The blade mechanism is preferably a bone cutting wire, and the cartridge further includes a wheel, gear mechanism, or any mechanism by which the blade mechanism is operable to cut the bone. The cartridge is preferably removable from the distal end of the tool so as to be replaceable after a surgical procedure, and preferably includes a wheel which engages the gear mechanism in such a fashion that when the handle, and consequently the rod, is rotated, the wheel rotates to provide movement to the wire bone cutting saw.

The tool further has an angled distal end which facilitates alignment of the cutting blade with the particular portion of the vertebra of the patient that is to be divided to allow the wire bone cutting saw to interact and divide the bone, and also includes a rounded ledge on the under surface beneath the exposed portion of the wire bone cutting saw to protect bone structures that are not intended to be cut from the saw. Preferably, the angle is determined by the portion of the vertebra to be cut. That is, when a tool is used to cut the lamina, the angle is less than the angle on a tool used to cut the facet joints. In addition, a tool used to cut the transverse processes would have an angle that is greater than the angle on a tool used to cut the facet joints. It is contemplated that a set of tools would be provided in a kit to allow the surgeon greater flexibility during the surgical procedure. It is also contemplated that the distal end of the tool is articulating, to allow the surgeon the flexibility to adjust the angle for his particular needs.

The handle at the surgeon's end of the device can be a wing-nut type handle, a spherical handle or any other ergonomically favorable geometric form to allow the surgeon to manually rotate the rod contained within the elongated shaft of the instrument. The shaft can be round, oblong, square, or multifaceted shape as viewed in the transaxial plane and has an outer diameter which is suitable to the procedure. That is, the tool can be designed for open-type surgical procedures, or can be designed for use in minimally-invasive laparoscopic procedures. The inner rod translates the rotation of the handle on the surgeon's end into rotation of the gear mechanism at the distal end of the instrument.

The invention provides for a set of the above-disclosed devices which can be precisely and safely guided to the vertebra. In one embodiment, the tool is advanced into the coronal plane of the lamina, and is used to create an osteotomy through the cancellous portion of the lamina and divides the lamina so that the posterior aspect of the lamina can be elevated while still attached to the musculature and periosteum. The musculature and periosteum provides a vascular supply to this bone; hence, vascularized autograft can be provided to the fusion bed in the setting of a posterior or posteriolateral fusion of the spine. In another embodiment, the tool is used to effectively divide the facet joints, and in another embodiment, is used to divide the transverse processes in the coronal plane. The division of the facet joints and the transverse processes in the coronal plane and through the cancellous portion of the transverse processes will retain the connection of the musculature and periosteum to each of the divided components. This will allow for vascularized autograft to be provided to the fusion bed in the setting of a posteriolateral fusion of the spine.

In the surgical procedure, the operating surgeon creates an incision in the patient in the midline overlying the segments of the spine to be fused. Using standard surgical techniques, the incision is extended down through the tissues underlying the skin until the spinous processes are exposed. However, in contrast to the standard exposure of a spine during spine fusion, the lamina, facets and transverse processes are not exposed, but the muscles and ligaments are permitted to remain attached to these structures. Blood flow to these areas is delivered in part through the muscles and periosteum. By retaining the patency of these structures, blood flow can be maintained to bone upon which osteotomies are performed.

After exposing the base of the spinous processes, the spinous processes are removed using standard surgical techniques. If the lamina is to be utilized for the vascularized autograft, a trough is created in the base of the lamina to allow for the introduction of the inventive tool. The surgeon introduces the device in such a manner that the opened end exposing the bone saw is in contact with the cancellous bone found in the mid position of the coronal plane of the lamina. The curve of the device facilitates placement herein and allows the surgeon to more precisely guide the instrument. Placement and guidance of the instrument is accomplished by maintaining a grasp on the non-rotating outer shaft of the device. The bone saw is engaged and deployed and an osteotomy can be carried from the midline laterally elevating the posterior portion of the lamina while still being adhered and connected to the musculature and periosteum. This is accomplished bilaterally at each level of the proposed fusion.

If the facets are to be utilized for the vascularized autograft, a tool to divide the facets is applied. This device, having an angle favorable to insertion around the facets is brought into position. Preferably, the distal end of the tool is provided with small projections extending therefrom. These projections are designed in such a fashion that they may be fitted over the superior and inferior aspects of the facet joints, thus stabilizing the device as the osteotomy for the facet joints is completed. The posterior aspect of the facet joint still connected to the musculature and periosteum can then be reflected posteriorly. This preserves the facet arteries to provide additional vascular supply to the fusion bed.

If the transverse processes are to be utilized for the vascularized autograft, the surgeon then exposes the base of the transverse process. A tool to divide the transverse process is the advanced to the portion to be cut, and is preferably provided with projections that can be fitted over the superior and inferior aspects of the transverse process. The transverse process is divided through the cancellous portion in the coronal plane.

The coronal osteotomies described above also provide an entry point to the pedicle should the surgeon wish to utilize pedicle screw fixation. The vascularized autograft, suspended on the muscles, is then mobilized into position in the intervertebral space to promote bone fusion.

### BRIEF DESCRIPTION OF THE DRAWINGS

The above and other objects, features and advantages will become more readily apparent from the following detailed description of preferred embodiments, accompanied by the following drawings, in which:

FIG. 1 is a perspective view of a first embodiment of the tool of the present invention with the blade cartridge removed;

FIG. 2 is a perspective view of the tool showing the undersurface of the blade cartridge, with the gear housing cover removed;

FIG. 3 is a perspective view of the tool of FIG. 1 in use;

FIG. 4 is an elevational view of the underside of the blade cartridge;

FIG. 5 is a perspective view of an alternative embodiment of the tool, with the blade cartridge and the gear housing cover removed;

FIG. 6 is a perspective view of another alternative embodiment of the tool; and

FIG. 7 is a partial exploded perspective view of the distal end of the tool of FIG. 6.

### DETAILED DESCRIPTION OF THE PREFERRED EMBODIMENTS

Preferred embodiments of the present invention will be described in detail hereinafter with reference to the accompanying drawings, in which the same reference numerals represent similar or identical element throughout the views. In addition, a detailed description of known functions and configurations will be omitted when it may obscure the subject matter of the present invention.

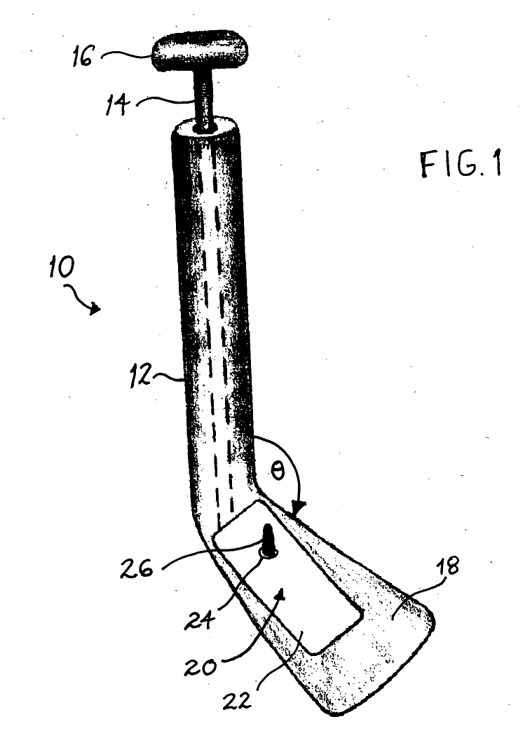

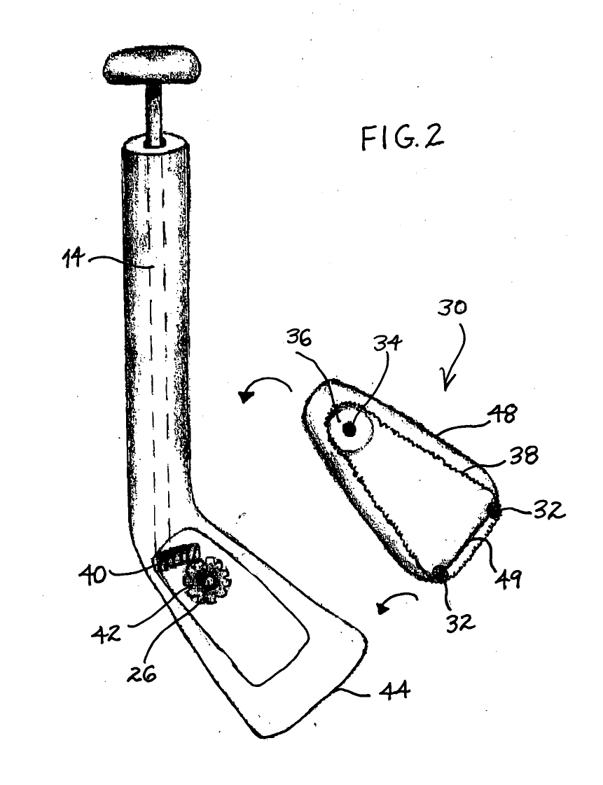

Turning now to the drawings, FIG. 1 shows a tool 10 according to the present invention. Tool 10 consists of an elongated shaft 12 having a coaxially disposed rod 14 passing therethrough. A handle 16 is disposed at a proximal end of the tool 10, and is operably engaged with rod 14, such that actuation of handle 16 effects movement of rod 14. A footplate 18 extends from shaft 12 at an angle which is determined according to the intended use of the tool 10. That is, the angle of the tool 10 is designed to align itself with the angle of the portion of the vertebra which it is intended to cut. For example, when tool 10 is use to create an osteotomy of the lamina of the vertebra, the angle θ is preferably approximately 120°. Furthermore, when the tool 10 is used to create an osteotomy of the facets, the angle θ is preferably approximately 135°. In addition, when the tool 10 is used to create an osteotomy of the transverse processes, the angle θ is preferably approximately 75°. Rod 12 extends into gear housing 20, which encloses a gear assembly preferably consisting of rod gear 40 and cartridge gear 42, as seen in FIG. 2. Gear post 26 is connected to and extends from cartridge gear 42 through hole 24 in cover 22, which encases the gear assembly and protects the gear assembly during use of tool 10. Preferably, rotation of handle 16 translates into rotation of rod 14, which turns rod gear 40. Rod gear 40 in turn causes rotation of cartridge gear 42, along with gear post 26.

As shown in FIG. 2, a blade cartridge 30 is removably attached to tool 10 at footplate 18. The underside of the cartridge 30 is shown in FIG. 2 to illustrate the blade mechanism. Cartridge 30 consists of a cover 48, which houses wheel 34 and wire guides 32, around which is positioned bone cutting wire 38. Bone cutting wire 38 extends a short distance beyond the end 49 of the cover 48, as well as beyond the end of the end 44 of the footplate 18, to facilitate cutting of the bone. End 44 of footplate 18 preferably serves as a retractor surface, enabling the surgeon to separate and move the divided bone after cutting. When fully assembled, cover 22 is in place on footplate 18 as shown in FIG. 1. Cartridge 30 is then positioned over footplate 18 and secured in any conventional manner such as snap fit, friction fit, screws, etc., so that gear post 26 fits into gear post hole 34 in wheel 36. Preferably, the inner surface of gear post hole 34 has gear teeth which mesh with gear teeth on gear post 26, to translate rotation of gear post 26 into rotation of wheel 34, which in turn will move bone cutting wire 38. Reciprocal movement of handle 16 will move wire 38 back and forth over the bone to cut the bone.

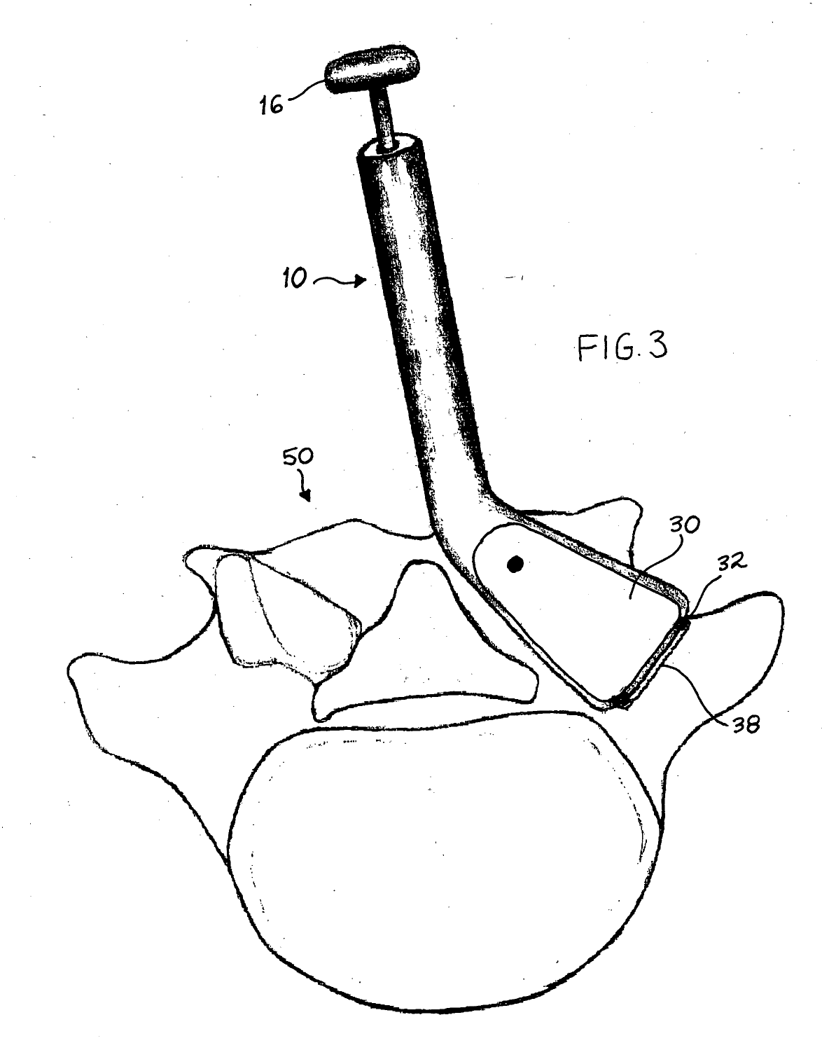

FIG. 3 illustrates the tool 10 in use during an osteotomy of the lamina of vertebra 50. Tool 10 is advanced to the lamina of the vertebra, and as seen in FIG. 3, a lamina cutting embodiment is chosen, with the footplate 18 disposed at an approximate angle of 120° with respect to the shaft 12. Cartridge 30 is in place on footplate 18, and bone cutting wire 38 extends over wire guides 32 and beyond retractor surface 44 at the end of the footplate 18. In use, the surgeon rotates or reciprocates handle 16 in a back and forth motion to move wire 38 to cut the bone.

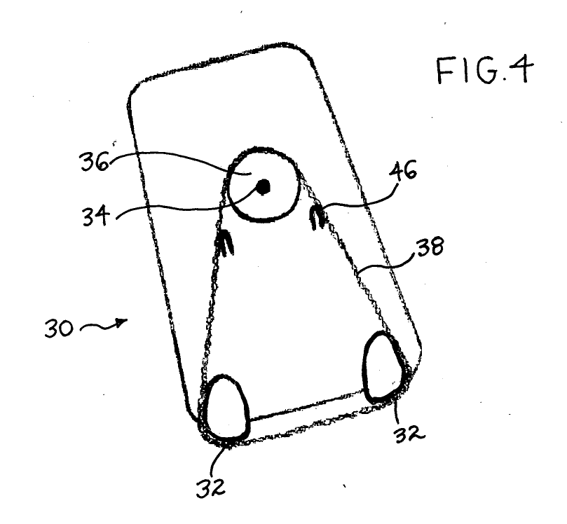

FIG. 4 shows an elevational view of the underside of tool 10. Secondary wire guides 46 are provided to facilitate movement of wire 38, which, as described above, moves over wire guides 32 due to the movement of wheel 36. It is also contemplated that wheel 36 can be provided directly on gear post 26, so that cartridge 30 is fit over the post and wire 38 is fit over the wheel during attachment of the cartridge.

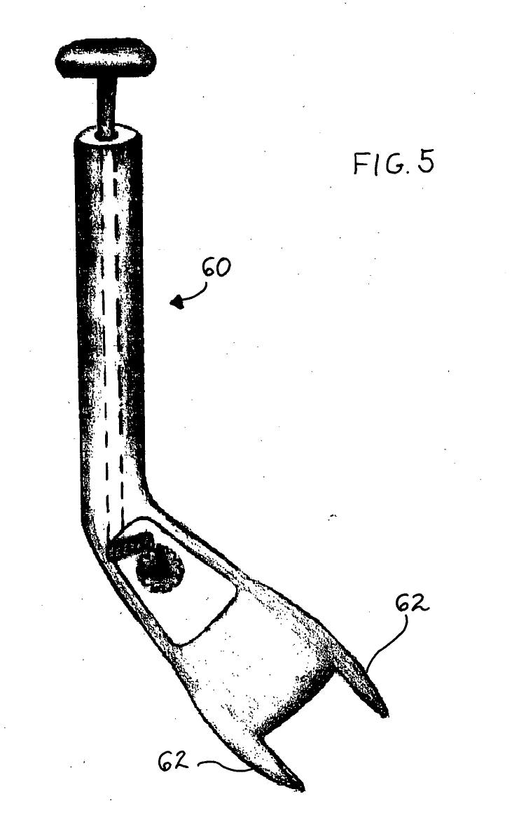

FIG. 5 shows an alternative embodiment of the tool, particularly suited for use in performing an osteotomy of the facets and the transverse processes. Tool 60 is similar to tool 10 except for the provision of guide prongs 62, which are designed to enclose the facet bone to accurately align the tool 60 with the bone to be cut. Preferably, the footplate extends from the shaft 12 at an angle of approximately 135° for a tool 60 that is to be used to perform an osteotomy of the facets, and at an angle of approximately 75° for a tool 60 that is to be used to perform an osteotomy of the transverse processes.

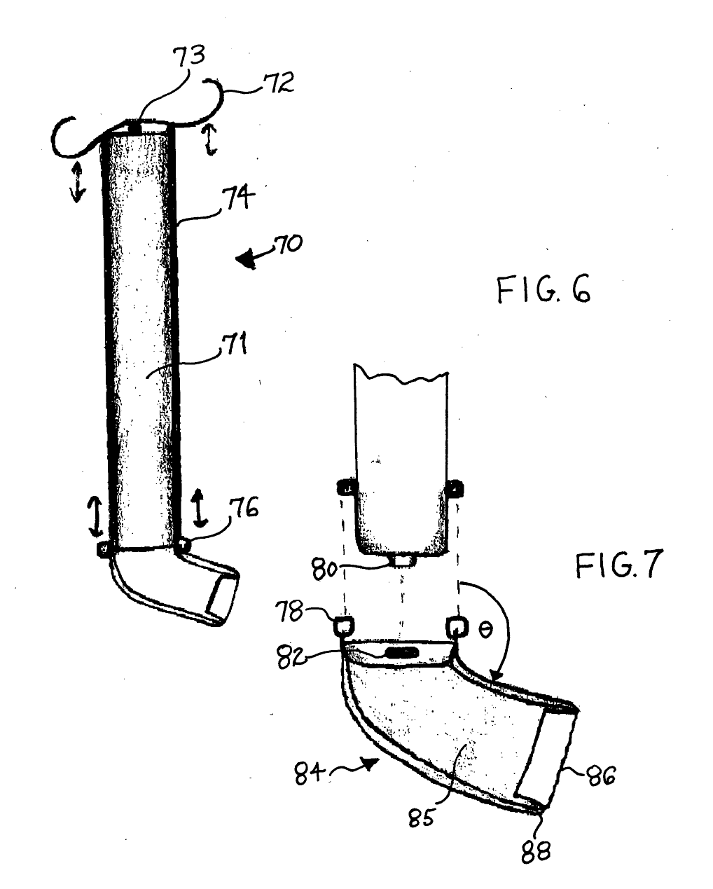

FIGS. 6 and 7 illustrate an alternative embodiment of the tool, which utilizes an up and down reciprocal motion of handle 72 to translate movement of the handle into reciprocal movement of the wire 86 to perform an osteotomy. Tool 70 consists of a shaft 71 having side rods 74 attached to opposite sides of handle 72, which pivots about pivot 73, and moves side rods 74 in an up and down manner. Side rods 74 terminate at rod tabs 76, at an end of shaft 71 that accommodates detachable footplate/cartridge 84. Footplate/cartridge 84 includes a body 85 having an alignment slot 82, which couples with alignment tab 80 on the distal end of shaft 71. A bone cutting wire 86 is provided, and is secured to wire tabs 78, which engage and cooperate with rod tabs 76. Preferably, wire 86 extends through body 85 and exits body 85 at prongs 88, and extends between the two prongs 88.

In use, handle 72 is rocked back and forth, which moves side rods up and down with respect to shaft 71. This in turn causes rod tabs to reciprocate alternatingly in an up and down manner, thus moving wire tabs 78 in a like manner. This will causes wire 86 to move back and forth between prongs 88 to cut the bone.

It is contemplated that the footplate/cartridge 84 be constructed with different angles, as described above, so that a particular footplate/cartridge 84 may be chosen depending on the portion of the vertebra that is to be cut. For example, a footplate/cartridge 84 having an angle of approximately 120° would be chosen and attached to the shaft 71 when the surgeon desires to perform an osteotomy on the lamina, while a footplate/cartridge 84 having an angle of approximately 135° would be chosen for osteotomies of the facets. A footplate/cartridge 84 having an approximate angle of 75° would be chosen when the transverse processes are to be cut.

While the present invention has been shown and described with reference to certain preferred embodiments thereof, it will be understood by those skilled in the art that various changes in form and detail may be made therein without departing from the spirit and scope of the present invention, as defined by the appended claims. For example, the handle of the tool may be operated by motor, and various types of bone saws may be utilized with the removable cartridge.

1. A tool for performing osteotomies of vertebra of the spine, comprising:
an elongated shaft;
an elongated rod coaxially disposed within the shaft;
a handle disposed at a proximal end of the rod;
a footplate extending at an angle from the shaft at a distal end of the shaft;
a gear assembly disposed within the footplate and engaging the rod; and
a removable blade assembly attached to the footplate and engaging the gear assembly;
wherein movement of the handle translates into movement of a blade of the blade assembly.
